SEQUENCE LISTING (16 sequences)

SEQ ID NO: 1 - DNA, 52 bp, Adenovirus
tgaaggggggctataaaagggggtgggggcgcgttcgtcctcactctcttcc

SEQ ID NO: 2 - DNA, 52 bp, Adenovirus
ggccgggggggtataaaagggggcgggccgctgctcgtcttcactgtcttcc

SEQ ID NO: 3 - DNA, 52 bp, Adenovirus
cgccgggggggtataaaagggggcggacctctgttcgtcctcactgtcttcc

SEQ ID NO: 4 - DNA, 52 bp, Adenovirus
tggtggtgggctataaaaaggggcgggtccttggtcttcatcgctttcttct

SEQ ID NO: 5 - DNA, 52 bp, Adenovirus
gtgcgtgggtgtataaaagggggcgtgtccgggctcttcatcactttcttcc

SEQ ID NO: 6 - DNA, 52 bp, Adenovirus
gggcggggggcgataaaagggggcggcgccgtcgtcgccgtcactgtcctct

SEQ ID NO: 7 - DNA, 11 bp, Artificial Sequence
  Description of Artificial Sequence Hypothetical “MAZ Consensus”
grggmggggmk

SEQ ID NO: 8 - DNA, 11 bp, Artificial Sequence
  Description of Artificial Sequence Hypothetical “MLP-36 GC Consensus”
kgscggggggg

SEQ ID NO: 9 - DNA, 12 bp, Artificial Sequence
  Description of Artificial Sequence Hypothetical “MLP-18 GC Consensus”
gggggcgggscc

SEQ ID NO: 10 - DNA, 10 bp, Adenovirus
kgggcggrry

SEQ ID NO: 11 - DNA, 229 bp, Adenovirus
actctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgc

SEQ ID NO: 12 - DNA, 52 bp, Adenovirus; identical to SEQ ID NO: 1

SEQ ID NO: 13 - DNA, 52 bp, Adenovirus
tgaaggggggctataaaagtgtgtgtgtgtgtgttcgtcctcactctcttcc

SEQ ID NO: 14 - DNA, 52 bp, Adenovirus
tgttgttgttctataaaagggggtgggggcgcgttcgtcctcactctcttcc

SEQ ID NO: 15 - DNA, 52 bp, Adenovirus
tgttgttgttctataaaagtgtgtgtgtgtgcgttcgtcctcactctcttcc

SEQ ID NO: 16 - DNA, 52 bp, Adenovirus
tgttgttgttctctccaagtgtgtgtgtgtgcgttcgtcctgaatctcttcc

(19) United States
(12) Patent Application Publication
(10) Pub. No.: US 2002/0055173 A1
(43) Pub. Date: May 9, 2002

(54) Method for preparing adenovirus vectors, vectors so prepared, and uses thereof

(76) Inventors: Christopher L. Parks, Boonton, NJ (US); Thomas Shenk, Princeton, NJ (US)

Correspondence Address: KLAUBER & JACKSON, 411 HACKENSACK AVENUE, HACKENSACK, NJ, 07601

(21) Appl. No.: 09/861,975

(22) Filed: May 21, 2001

Related U.S. Application Data
(63) Continuation of application No. 09/200,342, filed on Nov. 25, 1998, now abandoned.
(60) Provisional application No. 60/066,295, filed on Nov. 25, 1997.

(51) Int. Cl. A61K 48/00; C12N 15/861

(52) U.S. Cl. 435/456; 435/320.1; 424/93.2

(57) ABSTRACT

Multiple binding sites for the transcription factors MAZ and Sp1 within the adenovirus type 5 major late promoter have been identified by DNase I protection studies. In the proximal region of the promoter, both MAZ and Sp1 interact with GC-rich sequences flanking the TATA box. Two MAZ binding sites are centered at −18 and −36 relative to the transcriptional initiation site. Sp1 bound only to the −18 GC-rich sequence. Several sites of interaction were also evident in the distal region of the promoter. Both MAZ and Sp1 interacted with a sequence centered at −166, and MAZ bound weakly to an additional site centered at −130. Over expression of MAZ or Sp1 activated expression from the major late promoter in transient expression assays. Mutational analysis of the GC-rich sequences in the major late promoter suggested that a primary target of MAZ activation is the GC rich sequences flanking the TATA sequence, whereas Sp1 requires the distal GC-rich sequence elements to stimulate gene expression. This activation is enhanced by the adenovirus E1 A protein, and evidence for interaction between E1 A and both transcription factors was obtained using an immunoprecipitation assay. Activation by MAZ and Sp1 also was observed in transfection studies using the complete adenovirus type 5 genome as the target. Increased levels of late mRNA from both the L1 and L5 regions were observed when MAZ or Sp1 expression plasmids were transfected with viral DNA. Unexpectedly, activation of the major late promoter by MAZ and Sp1 was detected irrespective of whether the viral DNA could replicate.

The research leading to the instant Application was supported in part by National Cancer Institute Grant No. CA38965. The Government may have certain rights in the invention.

### TECHNICAL FIELD OF THE INVENTION

The present invention relates generally to the preparation of vectors and more particularly to the preparation of adenovirus vectors, to the preparation of virus particles by means of the vectors, and to the preparation of cells containing such vectors as by the transfection of such cells with the vectors to insert a particular DNA of interest. The invention makes use of the transcription factors MAZ and Sp1 to activate the adenovirus major late promoter (MLP). Activation of the MLP, in turn, allows for the replication, amplification, and encapsidization of a vector containing the two terminal segments of the adenovirus genome which flank any inserted non-adenovirus DNA. Therefore, this invention also relates to a system for the in vivo expression of therapeutic proteins, antisense RNA, and ribozymes, the coding sequence of which are flanked by the above-mentioned adenovirus genome sequences in a vector.

### BACKGROUND OF THE INVENTION

The adenovirus major late promoter (MLP) controls expression of the major late transcription unit that encodes most of the viral structural proteins and several nonstructural proteins (reviewed in 22). The MLP is active during both early and late periods of infection but reaches maximal activity after the onset of DNA replication. Genetic and biochemical studies have identified a number of transcription factor binding sites and corresponding DNA-binding proteins that regulate expression from the MLP. These include the TATA box binding protein (TBP) and the TFIID complex that bind the TATA element, the USF/MLTF binding site at −50, a CAAT box near −70, an initiator site at +1, and downstream elements that bind to a protein complex that includes cellular factors and the viral IVa2 protein (reviewed in 22). Most of these factor binding sites are conserved in the MLP of divergent adenovirus serotypes enforcing the conclusion that these sites are important for appropriate transcriptional regulation (FIG. 1 and ref. 25).

An interesting architectural feature of the MLP is the presence of GC-rich sequences surrounding the TATA box (FIG. 1). These sequences are well conserved in human adenoviruses as well as some other adenoviruses (FIG. 1 and ref. 25) which would imply a functional importance of the sequences to the MLP. Although the GC-rich elements can be extensively substituted with AT base pairs without inhibiting activity of the major late promoter in a whole cell extract (29), mutations in the upstream TATA-proximal GC-rich element reduced the activity of the MLP in virus-infected cells (3). Further, Yu et al. (30) found that the TATA-proximal GC-rich sequences formed nuclease-sensitive structures when the MLP was present in supercoiled plasmid DNA, but the physiological significance of this observation is not clear.

We have been interested in the transcription regulation of GC-rich promoters by the zinc-finger proteins MAZ and Sp1 (20). Since the GC-rich sequences in the MLP are potential binding sites for MAZ and Sp1, the ability of these factors to interact with the promoter and regulate its activity has been examined. The results as demonstrated herein, suggest that both factors can interact with the GC-rich sequences in the MLP, stimulate MLP activity and respond to the E1A protein.

### SUMMARY OF THE INVENTION

In its broadest aspect, the invention relates to the preparation of adenovirus vectors, and particularly, such vectors as are capable of replication on their own by the overexpression of two cellular transcription factors.

This invention provides a helper adenovirus vector comprising an adenovirus genome having a deletion of the nucleic acid of the origin of replication and the packaging sequence genes of the adenovirus genome. In one embodiment the vector further comprising a deletion of the E1A gene. In another embodiment the vector further comprises a deletion of the E1B gene. In another embodiment the vector further comprising an insertion of one or more nucleic acids of transcription factors within a region of the adenovirus genome. In one embodiment the transcription factors is MAZ and/or SP1.

This invention provides a pharmaceutical composition comprising: a) an adenovirus vector comprising the terminal segments of a linear adenovirus genome and a nucleic acid inserted between the terminal segments of the linear adenovirus genome, wherein the terminal segments comprise nucleic acids of the origin of replication and the packaging sequence genes of the adenovirus genome; b) a helper adenovirus vector comprising an adenovirus genome having a deletion of the nucleic acid of the origin of replication and the packaging sequence genes of the adenovirus genome; and c) a vector comprising one or more nucleic acids of a transcription factor, and a suitable diluent of carrier.

This invention provides a method of activating adenovirus major late promoter comprising transfecting a cell with: a) an adenovirus vector comprising the terminal segments of a linear adenovirus genome and a nucleic acid inserted between the terminal segments of the linear adenovirus genome, wherein the terminal segments comprise nucleic acids of the origin of replication and the packaging sequence genes of the adenovirus genome; b) a helper adenovirus vector comprising an adenovirus genome having a deletion of the nucleic acid of the origin of replication and the packaging sequence genes of the adenovirus genome; and c) a vector comprising one or more nucleic acids of a transcription factor, thereby activating the adenovirus major late promoter. In one embodiment the transcription factors is MAZ and/or SP1. In another embodiment the method further comprises transfecting the cell with a vector comprising nucleic acid which encodes an E1A gene.

This invention provides a method of preparing virus particles containing a nucleic acid encoding protein of interest comprising transfecting a cell with a) an adenovirus vector comprising the terminal segments of a linear adenovirus genome and a nucleic acid inserted between the terminal segments of the linear adenovirus genome, wherein the terminal segments comprise nucleic acids of the origin of replication and the packaging sequence genes of the adenovirus genome; b) a helper adenovirus vector comprising an adenovirus genome having a deletion of the nucleic acid of the origin of replication and the packaging sequence genes of the adenovirus genome; and c) a vector comprising one or more nucleic acids of a transcription factor, thereby preparing the virus particles. In one embodiment the transcription factors is MAZ and/or SP1. In another embodiment the method further comprises transfecting the cell with a vector comprising nucleic acid which encodes an E1A gene. In another embodiment the cell is a human cell.

This invention provides a gene therapy method comprising administering to a subject a pharmaceutical composition comprising: a) an adenovirus vector comprising the terminal segments of a linear adenovirus genome and a nucleic acid inserted between the terminal segments of the linear adenovirus genome, wherein the terminal segments comprise nucleic acids of the origin of replication and the packaging sequence genes of the adenovirus genome; b) a helper adenovirus vector comprising an adenovirus genome having a deletion of the nucleic acid of the origin of replication and the packaging sequence genes of the adenovirus genome; and c) a vector comprising one or more nucleic acids of a transcription factor, and a suitable diluent or carrier, thereby inserting the gene into the subject. In one embodiment the transcription factors is MAZ and/or SP1. In another embodiment the method further comprises administering a pharmaceutical composition comprising nucleic acid which encodes an E1A gene.

The present invention naturally contemplates several means for preparation of vectors containing the gene encoding the desired therapeutic protein, the vectors carrying the helper DNA sequences, and the vectors carrying the MAZ and/or Sp1 genes, including as illustrated herein known recombinant techniques, and the invention is accordingly intended to cover such synthetic preparations within its scope.

Likewise, the present invention extends to the preparation of virus particles capable of expressing proteins of interest when inserted in appropriate host cells, and to gene therapy techniques that achieve the direct introduction of such contructs into cells for therapeutic purposes.

Other uses and advantages of the present invention will become apparent to those skilled in the art from a review of the ensuing description which proceeds with reference to the following illustrative drawings.

### BRIEF DESCRIPTION OF THE DRAWINGS

FIG. 1. Alignment of adenovirus MLP sequences. For comparison, four sequence motifs from the MLPs are outlined including the TATA motif, initiator sequences and the GC-rich sequences (−36GC and −18GC) flanking the TATA box. At the bottom of the figure, consensus binding sites for MAZ (19) and Sp1 (12) are compared to the GC-rich consensus sequences flanking the TATA motif in the MLP.

FIG. 2. Analysis of DNA-protein interactions in the MLP by DNAse I protection. (A) Increasing amounts of MAZ protein were incubated with an MLP fragment spanning nucleotides +47 to −260 relative to the start site that was [³²P] end-labeled at nucleotide +47. After limited digestion with DNAse I, the footprint reaction products were processed and electrophoresed in a sequence gel next to a GA sequencing ladder. Bars at the sides of the autoradiograms highlight the regions of protection. The black bar represents strong MAZ binding sites and the grey bar represents weaker MAZ binding sites. Nucleotide position relative to the start site is indicated beside the autoradiogram. (B) The experiment shown in this panel was performed as described above, but also included footprint reactions containing Sp1 protein. Sp1 footprints are highlighted with a hatched bar. (C) Summary of footprinting experiments that show the binding sites for MAZ and Sp1. Data was scanned an cropped using Ofoto software, and figures were prepared using Canvas 3.5 software.

FIG. 3. Activation of the MLP by MAZ, Sp1, and E1A. (A) Cotransfection experiments assessing the ability of MAZ, Sp1 and E1A to activate a MLP-luciferase reporter plasmid. The MLP-luciferase construct contained MLP sequences from −260 to +10. Hela cells were transfected with reporter plasmid (10 μg), and various effector plasmids: pCMV-E1A (1 μg), PCMV-MAZ (10 μg) or pCMV-Sp1 (10 μg). When necessary the CMV expression vector with no insert was included to maintain a constant quantity of CMV promoter-containing plasmid. The results are expressed as the level of activation achieved relative to the activity obtained when the expression plasmid with no inserted effector sequence was included. The bar graph presents the mean levels of activation along with standard deviations calculated from five independent experiments. (B) Western blot analysis monitoring expression of the epitope-tagged MAZ and Sp1 proteins in transfected cells. The products of the expression plasmids are indicated above each lane; vector designates cells receiving the empty expression plasmid. The sizes in kilodaltons of marker proteins is indicated to the right of the autoradiogram. (C) Analysis of luciferase RNA produced in cells transfected as in part A. The RNA was hybridized to the MLP-luciferase probe DNA depicted above the autoradiogram. Hybridization was terminated by digestion with S1 nuclease and the digestion products were electrophoresed in a denaturing polyacrylamide gel. The MLP-specific signal is indicated by an arrow, and the sizes of marker DNAs are indicated. (D) Immunoprecipitation assays from extracts of cells transfected as in part A. The protein expression plasmid used in each transfection is indicated above the lanes in the autoradiogram. In the upper panel, the immunoprecipitations were performed with a monoclonal antibody specific for the flu epitope-tag (a-flu tag IP); immunoprecipitated proteins were processed for Western blotting, again using the monoclonal antibody specific for the flu epitope-tag (a-flu tag blot). In the right-side panel an identical set of immunoprecipitated proteins was probed in a Western blot using a monoclonal antibody to the E1A protein (a-E1A blot).

FIG. 4. Effect of mutations in the GC-rich sequences flanking the TATA motif on MAZ and Sp1 binding. (A) Sequence of the wild-type minimal MLP and its mutant derivatives. (B) DNase I footprint analysis was performed to assay MAZ (B) and Sp1 (C) binding to wild-type and mutant MLPs. The probe DNA was 5′ end-labeled in the luciferase coding region. The strong (black) and weak (grey) MAZ footprints and the Sp1 footprint on wild-type DNAs are designated by bars on the side of the autoradiogram. Sequence positions relative to the start site are shown next to the GA sequence reaction.

FIG. 5. Effect of MLP mutations on the activity of the minimal MLP. Luciferase reporter plasmids were prepared with the minimal promoter fragments shown FIG. 4A. (A) The in vitro transcription activity of wild-type and mutant MLPs was assayed in a whole cell extract. Reaction products were analyzed by primer extension and denaturing polyacrylamide gel electrophoresis. The template DNAs used in the transcription reactions are indicated above the lanes in the autoradiogram. Migration of the 75 base marker (M) is indicated at the left and the MLP-specific band is marked by an arrow. (C) Transfection experiments employing wild-type and mutant MLP luciferase plasmids. Plasmids (0.2 μg) were transfected into 293 cells with effector plasmids (1 μg) expressing MAZ (grey bar) or Sp1 (hatched bar). Activation was calculated from seven independent experiments.

FIG. 6. Major late gene expression from transfected viral DNA. 293 cells were transfected with adenovirus DNA (10 μg) plus an expression plasmid (10 μg) producing the factor designated above each lane; vector indicates that the effector expression plasmid with no insert was included. Cells were harvested 48 h after transfection and total RNA was isolated. The RNA was hybridized to [³²P] end-labeled probed designed to detect the 5′ end of L1 RNAs (A) or RNA from the L5 region (B). The presence (+) or absence (−) of hyrdoxyurea during the 48 hr transfection period is indicated. The sizes of marker DNAs are indicated on the left side of the autoradiograms. Negative control RNA was prepared from mock-transfected cells and positive control RNA was isolated from cells infected with adenovirus at a multiplicity of 20 pfu/cell. (C) Replication of transfected adenovirus DNA. Viral DNA was harvested at 72 h after transfection by the Hirt procedure and analyzed by Southern blot. A [³²P] labeled riboprobe specific for the Ad5 HindIII-E fragment was used as the hybridization probe.

### DETAILED DESCRIPTION

In its broadest aspect, the invention relates to the preparation of adenovirus vectors, and particularly, such vectors as are capable of replication on their own by the overexpression of two cellular transcription factors that have been found to interact with the Adenovirus Major Late Promoter (MLP). Binding sites within the adenovirus major late promoter for two cellular transcription factors that interact with similar DNA sequences have been identified.. These transcription factors are termed MAZ and Sp1. As shown herein, over expression of MAZ or Sp1 can markedly induce the activity of the adenovirus major late promoter, that both factors interact with the adenovirus-coded E1A transcriptional activating protein, and that they cooperate with E1A protein to activate the major late promoter. When the complete adenovirus DNA is transfected into cells and adenovirus DNA replication is blocked by the addition of hydroxyurea, overexpression of MAZ or Sp1 enhances the accumulation of mRNA encoded by the L1 and L5 regions of the major late transcription unit. Enhancement of L5 RNA accumulation was unexpected because DNA replication is normally required for expression of this region of the viral genome.

As stated above, the observation that overexpression of MAZ or Sp1 can unexpectedly activate accumulation of L5 RNA suggests a scheme for the complementation of adenovirus vectors that can not replicate on their own. A vector DNA molecule would be prepared that contains short segments of DNA (several hundred base pairs) from the ends of the linear adenovirus chromosome; these terminal segments would include the adenovirus origins of DNA replication (needed to replicate and amplify the vector DNA molecule) and packaging sequence (needed to encapsidate the vector DNA molecule into a virus particle). Non-adenovirus DNA, e.g., DNA encoding a therapeutic protein, would be inserted between the two terminal segments of the adenovirus genome. In contrast to normal adenovirus, this adenovirus vector could not be propagated in human cells because it lacks all of the adenovirus genes that encode products needed for replication of viral DNA and its assembly into virus particles. A helper DNA molecule would be prepared that contains all of the adenovirus genome except the terminal sequences that are present in the vector molecule, and it would provide all of the trans-acting functions needed for replication and encapsidation of the vector DNA. The helper DNA itself can not be replicated and amplified since it lacks the replication origins; it can not be packaged into virus particles since it lacks the packaging sequence; and it can not efficiently recombine with the vector DNA since the two DNAs share no sequence in common, as would be needed for efficient, homologous recombination.

If the vector and helper DNAs are mixed and transfected into human cells where adenovirus can normally replicate, little or no vector particles will be produced because the helper DNA will not replicate and therefore will not express all of the gene products encoded within the major late transcription unit. Replication has been shown to be needed to activate full expression of the major late promoter and to induce the accumulation of the L3, L4, and L5 families of mRNAs encoded by the “downstream” portion of the major late transcription unit (reviewed in Shenk, 1996). However, as noted above, it has presently been discovered that over expression of MAZ or Sp1 unexpectedly can activate the accumulation of RNA from the downstream portion of the adenovirus major late transcription unit in the absence of DNA replication. Therefore, the results predict that if the vector and helper DNAs together with a plasmid encoding MAZ and/or Sp1 are transfected together into human cells where adenovirus can replicate, the vector DNA will be replicated and packaged into virus particles. The vector will replicate because MAZ and/or Sp1 will activate expression of the major late unit within the helper, even though the helper DNA does not replicate. The viral products encoded by the helper DNA will enable the vector DNA to replicate and to be packaged into virus particles. For the transfection approach to work well, a cell line must be used that can be very efficiently transfected. There are clones of 293 cells that fit this requirement.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

A “replicon” is any genetic element (e.g., plasmid, chromosome, virus) that functions as an autonomous unit of DNA replication in vivo; i.e., capable of replication under its own control. A “vector” is a replicon, such as plasmid, phage or cosmid, to which another DNA segment may be attached so as to bring about the replication of the attached segment.

A “DNA” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences. The promoter comprises a bacterial, yeast, insect or mammalian promoter. Example of promoters include: CMV, HMCV, SV40, and RSV.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

The phrase “therapeutically effective amount” is used herein to mean an amount sufficient to prevent, and preferably reduce by at least about 30 percent, more preferably by at least 50 percent, most preferably by at least 90 percent, a clinically significant change in the S phase activity of a target cellular mass, or other feature of pathology such as for example, elevated blood pressure, fever or white cell count as may attend its presence and activity.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

In its primary aspect, the present invention concerns the use of an adenovirus-based vector carrying a non-adenovirus-based DNA sequence for use in therapeutics. This vector contains two short segments of DNA (several hundred base pairs) from the ends of the linear adenovirus chromosome, which include the adenovirus origins of DNA replication (needed to replicate and amplify the vector DNA molecule) and packaging sequence (needed to encapsidate the vector DNA molecule into a virus particle), flanking any non-adenovirus DNA sequence. A helper DNA molecule, containing all of the adenovirus genome except for the terminal sequences that are present in the vector molecule, provides all of the trans-acting functions needed for replication and encapsidation of the vector DNA. In vivo or in vitro expression, or administration of MAZ, and/or Sp1, and/or E1A will activate the major late promoter of adenovirus, or any of the sequences of SEQ. ID NOs:1-15, which are contained in the helper DNA, thus causing the replication, amplification, and encapsidization of the vector containing the desired therapeutic DNA sequence.

In a particular embodiment, the present invention extends to gene therapy such that the invention describes a method for expressing any therapeutic protein or therapeutic antisense RNA sequence, or therapeutic ribozyme using the adenovirus constructs and transcription factors (MAZ Sp1, and E1A) described herein.

This invention provides an adenovirus vector comprising the terminal segments of a linear adenovirus genome and a nucleic acid inserted between the terminal segments of the linear adenovirus genome, wherein the terminal segments comprise nucleic acids of the origin of replication and the packaging sequence genes of the adenovirus genome. In one embodiment the adenovirus vector is an adenovirus type 5. As contemplated herein, the nucleic acid encodes a protein, an antisense RNA, or a ribozyme. The protein may be any therapeutic protein of interest. Further the vector comprises a selectable marker. The selectable marker is beta galactosidase or beta lactamase.

This invention provides a helper adenovirus vector comprising an adenovirus genome having a deletion of the nucleic acid of the origin of replication and the packaging sequence genes of the adenovirus genome. In one embodiment the vector further comprising a deletion of the E1A gene. In another embodiment the vector further comprises a deletion of the E1B gene. In another embodiment the vector further comprising an insertion of one or more nucleic acids of transcription factors within a region of the adenovirus genome. In one embodiment the transcription factors is MAZ and/or SP1. It is contemplated by this invention that the deletion of nucleic acid within region of the E1A and E1B gene may be a deletion of the entire nucleic acid sequence or a deletion which is sufficient to abrogate the function of the genes. MAZ and SPI means any and all analogs, fragments, homolgues, mutanst, or variants thereof which have the functional activity of MAZ and SP1, namely as transcription factors.

This invention provides a pharmaceutical composition comprising: a) an adenovirus vector comprising the terminal segments of a linear adenovirus genome and a nucleic acid inserted between the terminal segments of the linear adenovirus genome, wherein the terminal segments comprise nucleic acids of the origin of replication and the packaging sequence genes of the adenovirus genome; b) a helper adenovirus vector comprising an adenovirus genome having a deletion of the nucleic acid of the origin of replication and the packaging sequence genes of the adenovirus genome; and c) a vector comprising one or more nucleic acids of a transcription factor, and a suitable diluent of carrier.

This invention provides a method of activating adenovirus major late promoter comprising transfecting a cell with: a) an adenovirus vector comprising the terminal segments of a linear adenovirus genome and a nucleic acid inserted between the terminal segments of the linear adenovirus genome, wherein the terminal segments comprise nucleic acids of the origin of replication and the packaging sequence genes of the adenovirus genome; b) a helper adenovirus vector comprising an adenovirus genome having a deletion of the nucleic acid of the origin of replication and the packaging sequence genes of the adenovirus genome; and c) a vector comprising one or more nucleic acids of a transcription factor, thereby activating the adenovirus major late promoter. In one embodiment the transcription factors is MAZ and/or SP1. In another embodiment the method further comprises transfecting the cell with a vector comprising nucleic acid which encodes an E1A gene.

This invention provides a method of preparing virus particles containing a nucleic acid encoding protein of interest comprising transfecting a cell with a) an adenovirus vector comprising the terminal segments of a linear adenovirus genome and a nucleic acid inserted between the terminal segments of the linear adenovirus genome, wherein the terminal segments comprise nucleic acids of the origin of replication and the packaging sequence genes of the adenovirus genome; b) a helper adenovirus vector comprising an adenovirus genome having a deletion of the nucleic acid of the origin of replication and the packaging sequence genes of the adenovirus genome; and c) a vector comprising one or more nucleic acids of a transcription factor, thereby preparing the virus particles. In one embodiment the transcription factors is MAZ and/or SP1. In another embodiment the method further comprises transfecting the cell with a vector comprising nucleic acid which encodes an E1A gene. In another embodiment the cell is a human cell.

This invention provides a gene therapy method comprising administering to a subject a pharmaceutical composition comprising: a) an adenovirus vector comprising the terminal segments of a linear adenovirus genome and a nucleic acid inserted between the terminal segments of the linear adenovirus genome, wherein the terminal segments comprise nucleic acids of the origin of replication and the packaging sequence genes of the adenovirus genome; b) a helper adenovirus vector comprising an adenovirus genome having a deletion of the nucleic acid of the origin of replication and the packaging sequence genes of the adenovirus genome; and c) a vector comprising one or more nucleic acids of a transcription factor, and a suitable diluent or carrier, thereby inserting the gene into the subject. In one embodiment the transcription factors is MAZ and/or SP1. In another embodiment the method further comprises administering a pharmaceutical composition comprising nucleic acid which encodes an E1A gene.

Further, the vector may be administered in combination with other cytokines or growth factors include but are not limited to: IFN γ or α, IFN-β; interleukin (IL) 1, IL-2, IL-4, IL-6, IL-7, IL-12, tumor necrosis factor (TNF) α, TNF-β, granulocyte colony stimulating factor (G-CSF), granulocyte/macrophage CSF (GM-CSF); accessory molecules, including members of the integrin superfamily and members of the Ig superfamily such as, but not limited to, LFA-1, LFA-3, CD22, and B7-1, B7-2, and ICAM-1 T cell costimulatory molecules. It is contemplated by this invention that use of the adenovirus vector could be used similarly in conjunction with chemo- or radiotherapeutic intervention. DNA damaging agents or factors are known to those skilled in the art and means any chemical compound or treatment method that induces DNA damage when applied to a cell. Such agents and factors include radiation and waves that induce DNA damage such as, gamma -irradiation, X-rays, UV-irradiation, microwaves, electronic emissions, and the like.

In a preferred embodiment of this invention, 293 cells would be transfected and the helper DNA would lack the adenovirus E1A and E1B genes, which have oncogenic properties and are present and expressed in the adenovirus-transformed 293 cells. Even though the design of the system prevents the helper DNA from recombining with the vector DNA, it would be an added safety feature and asset to the vector system to separate the E1A and E1B genes from the helper so that two independent recombination events would be required to generate wild-type adenovirus during propagation of the vector.

In a further embodiment, variations in the vector propagation scheme are envisioned that would involve cloning the MAZ and/or Sp1 gene into the helper construct and using a helper virus rather than helper DNA.

In yet a further embodiment, expression of the adenovirus L4-100 kDa protein can be conducted from either from a plasmid or from within the genome of 293 cells since this protein has been shown to be needed for efficient translation of late viral mRNAs (reviewed in 31), and its constitutive expression might greatly enhance the production of proteins from mRNAs encoded by the helper virus.

In a further aspect, the present invention extends to the use of the genes encoding the transcription factors MAZ and Sp1, and their gene products for the purpose of activating the MLP of adenovirus. In a still further aspect, MAZ and Sp1 can be used to activate the MLP of helper DNA, as described supra, and thus stimulate the replication and encapsidization of adenovirus particles containing a vector (as described supra) that contains DNA encoding a therapeutic protein.

As used herein, “pharmaceutical composition” could mean therapeutically effective amounts of the vector together with suitable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts). solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

Further, as used herein “pharmaceutically acceptable carrier” are well known to those skilled in the art and include, but are not limited to, 0.01-0.M and preferably 0.05M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvant include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvant such as BCG (bacille Calmette-Guerin) and Corynebacterium parvum. Preferably, the adjuvant is pharmaceutically acceptable.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

When administered, compounds are often cleared rapidly from mucosal surfaces or the circulation and may therefore elicit relatively short-lived pharmacological activity. Consequently, frequent administrations of relatively large doses of bioactive compounds may by required to sustain therapeutic efficacy. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

Dosages. The sufficient amount may include but is not limited to from about 1 μg/kg to about 1000 mg/kg. The amount may be 10 mg/kg. The pharmaceutically acceptable form of the composition includes a pharmaceutically acceptable carrier.

The preparation of therapeutic compositions which contain an active component is well understood in the art. Typically, such compositions are prepared as an aerosol of the polypeptide delivered to the nasopharynx or as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

An active component can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

A composition comprising “A” (where “A” is a single protein, DNA molecule, vector, etc.) is substantially free of “B” (where “B” comprises one or more contaminating proteins, DNA molecules, vector, etc.) when at least about 75% by weight of the proteins, DNA, vector (depending on the category of species to which A and B belong) in the composition is “A”. Preferably, “A” comprises at least about 90% by weight of the A+B species in the composition, most preferably at least about 99% by weight.

The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carr The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalent, syringe, pipette, forceps, measured spoon, eye dropper or any such medically approved delivery vehicle.

The following example is presented in order to more fully illustrate the preferred embodiments of the invention. They should in no way be construed, however, as limiting the broad scope of the invention. This invention may be embodied in other forms or carried out in other ways without departing from the spirit or essential characteristics thereof. The present disclosure is therefore to be considered as in all respects illustrative and not restrictive, the scope of the invention being indicated by the appended Claims, and all changes which come within the meaning and range of equivalency are intended to be embraced therein. Various references are cited throughout this specification, each of which is incorporated herein by reference in its entirety.

### EXAMPLE 1

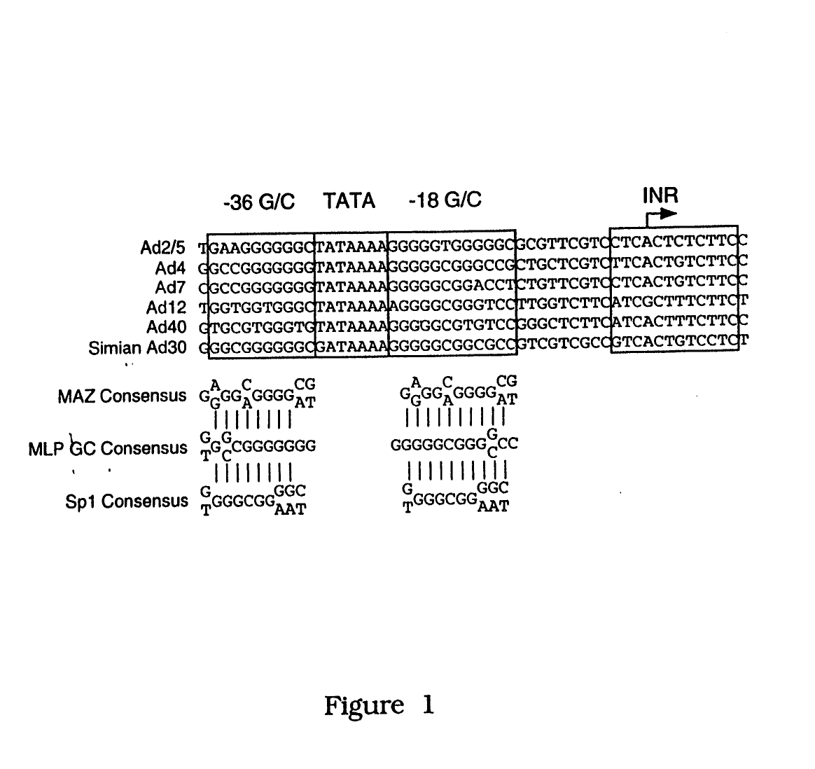

The adenovirus major late promoter (MLP) controls expression of the major late transcription unit that encodes most of the viral structural proteins and several nonstructural proteins (reviewed in 22). The MLP is active during both early and late periods of infection but reaches maximal activity after the onset of DNA replication. Genetic and biochemical studies have identified a number of transcription factor binding sites and corresponding DNA-binding proteins that regulate expression from the MLP. These include the TATA box binding protein (TBP) and the TFIID complex that bind the TATA element, the USF/MLTF binding site at −50, a CAAT box near −70, an initiator site at +1, and downstream elements that bind to a protein complex that includes cellular factors and the viral IVa2 protein (reviewed in 22). Most of these factor binding sites are conserved in the MLP of divergent adenovirus serotypes enforcing the conclusion that these sites are important for appropriate transcriptional regulation (FIG. 1 and ref. 25). An interesting architectural feature of the MLP is the presence of GC-rich sequences surrounding the TATA box (FIG. 1). These sequences are well conserved in human adenoviruses as well as some other adenoviruses (FIG. 1 and ref. 25) which would imply a functional importance of the sequences to the MLP. Although the GC-rich elements can be extensively substituted with AT base pairs without inhibiting activity of the major late promoter in a whole cell extract (29), mutations in the upstream TATA-proximal GC-rich element reduced the activity of the MLP in virus-infected cells (3). Further, Yu et al. (30) found that the TATA-proximal GC-rich sequences formed nuclease-sensitive structures when the MLP was present in supercoiled plasmid DNA, but the physiological significance of this observation is not clear.

We have been interested in the transcription regulation of GC-rich promoters by the zinc-finger proteins MAZ and Sp1 (20). Since the GC-rich sequences in the MLP are potential binding sites for MAZ and Sp1, the ability of these factors to interact with the promoter and regulate its activity has been examined. The results suggest that both factors can interact with the GC-rich sequences in the MLP, stimulate MLP activity and respond to the E1A protein.

### MATERIALS AND METHODS

### Plasmids, Viruses and Cells

Expression plasmids that produce flu epitope-tagged MAZ and Sp1 were previously described (20). The 289 amino acid residue E1A protein cDNA (13S E1A) was expressed from the CMV promoter (23). An epitope-tagged YY1 expression plasmid was prepared by inserting the YY1 cDNA into plasmid pRep4 (InVitrogen). The MLP construct (pMLP −260/+11) was prepared by cloning a DNA fragment generated by the polymerase chain reaction using Pfu DNA polymerase (Stratagene). The promoter fragment was cloned into the luciferase reporter plasmid pGL2-basic (Promega). Minimal MLP constructs contain sequences from −48 to +11 relative to the major late start site cloned into pGL2-basic. These were prepared by cloning double-stranded oligonucleotides into the luciferase vector. A plasmid that supplied hybridization probes for detection of major late L1 RNA 5′ ends was prepared by generating a cDNA that included the first leader and part of the second leader. This cDNA was fused to promoter sequences from −260 to +1 and cloned into vector pSP72 (Promega). A genomic DNA clone containing part of the L5 region was prepared by cloning the Ad5 DNA sequence from 89 to 92 map units into pGem4 (Promega).

The adenovirus type 5 (Ad5) El A-minus mutant, dl312 (11), was propagated in 293 cells which express the E1A protein (6), and viral DNA was prepared from purified virus as described earlier (19). Infections were performed at a multiplicity of 20 pfu/cell.

HeLa cells were maintained in Dulbecco's minimal essential medium supplemented with 10% Fetal Clone II serum (HyClone Laboratories). 293 cells were grown in Iscoves modified Dulbecco's medium (IMDM) supplemented with 10% fetal bovine serum (HyClone Laboratories).

### Expression Assays

HeLa and 293 cells were transfected by the calcium phosphate precipitation method, harvested and processed for luciferease assays as described earlier (20). A modified protocol was used when viral DNA was transfected into 293 cells (24). Viral DNA and expression plasmid were combined and the solution was adjusted to a final concentration of 0.3 M CaCl₂ in a total volume of 1 ml. To form the precipitate, 1 ml of hepes-buffered saline (2) was added to the DNA-calcium mixture and pipeted up and down five times to mix. The precipitate was allowed to form for 1 min and the entire 2 ml was distributed over a 10 cm plate of 293 cells containing 9 ml of IMDM supplemented with 10% fetal bovine serum. The precipitate was incubated with the cells for 12-16 h, then the cells were washed and fresh medium was added. Cells were harvested for RNA preparation at 44-48 h after the start of transfection. In cases where DNA replication was blocked with hydroxyurea (Calbiochem), the drug (10 mM) was added at 1 h after the start of the transfection and maintained in the medium until harvest.

Generally, RNA was prepared from transfected cells by guanidinium lysis and centrifugation through CsCl₂ (2). Several RNA preparations were made using the guanidinium/phenol extraction method (4) using Trizol reagent (Life Technologies). Nuclease S1 analysis was performed essentially as described earlier (20) with some modifications: RNA/DNA hybrids were digested with 1300 units S1 (Boehringer Mannheim) per ml; L5 DNA/RNA hybrids were digested at 30° C.; and nuclease digestion was performed for 1 h. Procedures for the preparation of end-labeled probes and hybridization conditions can be found in Parks and Shenk (20). The MLP 5′ end probe was labeled at a ScaI site (Ad5 nucleotide 7148). The L5 probe was labeled at a BgIII site (Ad5 nucleotide 32491). For detection of luciferase RNA, the MLP-luciferase plasmid DNA was labeled at the XbaI site in the luciferase coding region. Hybridizations were performed for 8-16 h at 47° C. (MLP 5′ end or luciferase probes) or 50° C. (L5 probe).

Immunoprecipitation of proteins from extracts of transfected cells was performed as described (2) using monoclonal antibody 12CA5 specific for the flu-epitope tag (14) and “E1A” buffer conditions (9). The Western analysis was performed as described earlier (20), and employed antibody to the flu-epitope tag or the M73 monoclonal antibody to the E1A protein (8).

To monitor viral DNA replication, cells were transfected as described above except that the calcium phosphate transfection mixture was scaled down 50% and 293 cells in 6 well plates were transfected with 2.5 mg Ad DNA and 5mg of the appropriate plasmid per well. DNA was harvested 48-72 hours after transfection using the modified Hirt procedure described by Volkert and Young (28). The DNA was digested with HindIII and analyzed by Southern blot (2). The blot was hybridized to a ³²P-labeled riboprobe complementary to Ad5 sequences 5788-6095.

### DNase I Footprinting and in vitro Transcription

Details of DNase I footprinting and in vitro transcription assays can be found in Parks and Shenk (20). Purified recombinant MAZ was prepared as described previously (20), and recombinant Sp1 was purchased (Promega). Crude whole cell extracts were prepared and used for in vitro transcription (15). The transcription reactions differed slightly from earlier studies by inclusion of pBluescript SK (Stratagene) as nonspecific DNA rather than poly dG/dC-dG/dC. RNA isolated from reaction mixtures was analyzed by primer extension (20) performed at 50° C. with Superscript II reverse transcriptase (Life Technologies).

### Results

### MAZ and Sp1 Bind at Multiple Sites Within the MLP

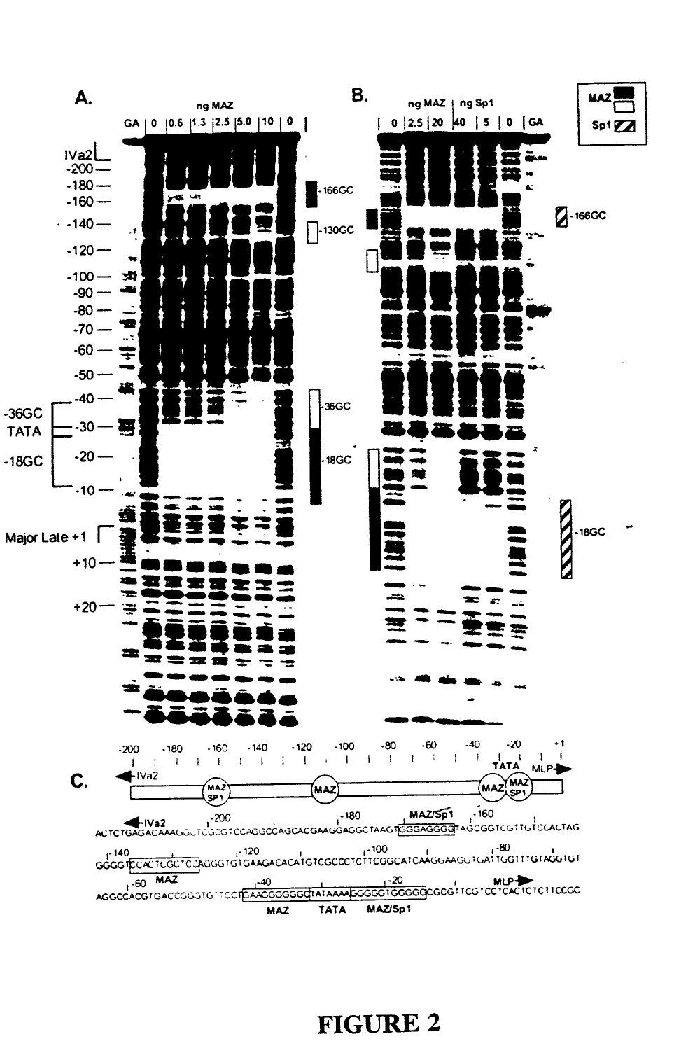

In interest in transcription factors MAZ and Sp1 (20) led us to examine the possibility that these factors might influence the activity of the MLP through GC-rich sequences centered at −18 (−18GC), −36 (−36GC) and −166 (−166GC) relative to the start of transcription. The site at −166 with respect to the MLP is positioned at −45 in the divergently transcribed IVa2 promoter. The −18GC and −36GC sequences (FIG. 1) were especially intriguing candidates for study because they flank the TATA motif, and they are conserved in a variety of adenoviruses. The ability of the −18GC and −36GC sequences to interact with MAZ and Sp1 was first tested. A footprint reaction revealed that MAZ binds to the MLP at multiple sites (FIG. 2A). Two sites are upstream of −100 and the remaining two sites coincide with the GC-rich sequences near the TATA box. Titration of the amount of MAZ added to the assay revealed the presence of two binding sites flanking the TATA box; the −18GC binding site is occupied at a lower protein concentration and at higher concentrations MAZ also binds to the lower affinity −36GC binding site. Sp1 interacted less extensively with the promoter than MAZ (FIG. 2B). In the distal region of the MLP, Sp1 binds only to the −166 site, and in the proximal promoter Sp1 binds only to the −18GC sequence.

### MAZ and Sp1 Cooperate With E1A to Activate the MLP

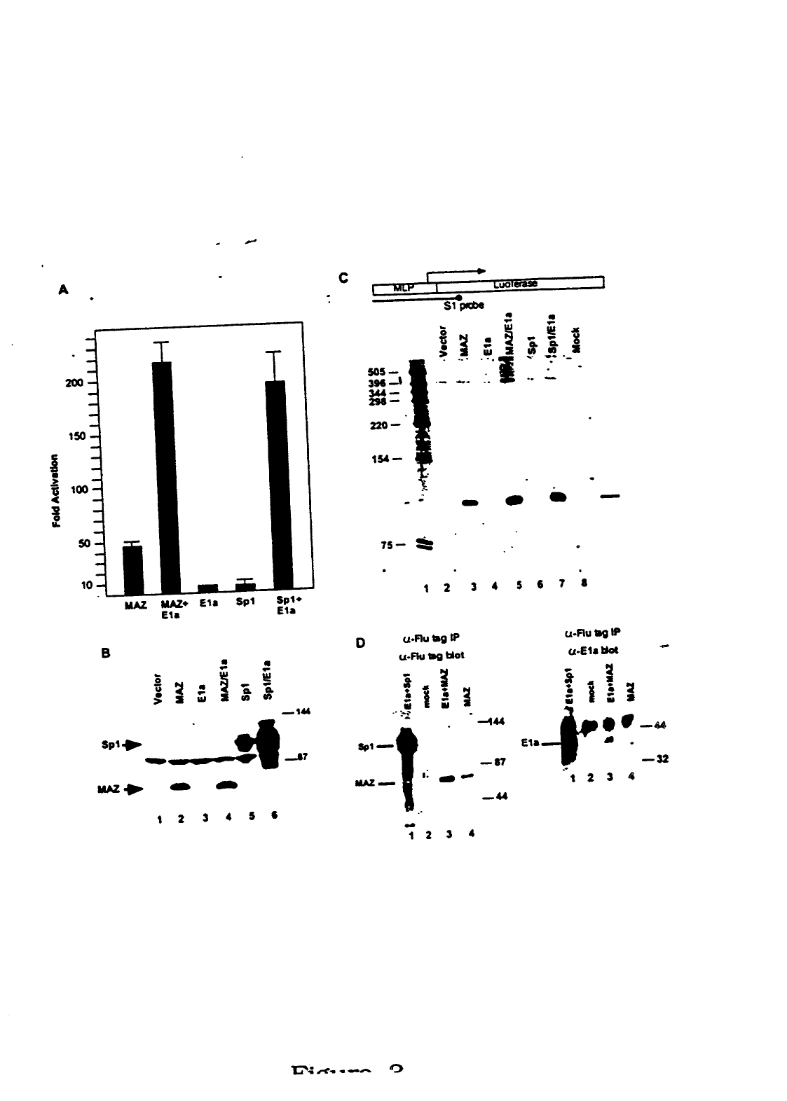

After establishing that the GC-rich sequences in the MLP interacted with MAZ and Sp1, whether the transcription factors affected the activity of the promoter was tested. To do this experiment, transient expression assays to examine the effect of over expression of MAZ or Sp1 on the activity of an MLP reporter plasmid was used. Sequences from −260 to +11 relative to the major late start site were cloned into a luciferase reporter plasmid, and this construct was cotransfected with expression plasmids that encoded epitope-tagged MAZ or Sp1. The effect of over expression of the 289 amino acid residue E1A activator protein encoded by the Ad5 13S mRNA was examined (reviewed in 22). MAZ increased luciferase activity by a factor of 40-50, whereas E1A or Sp1 provided a more modest increase of 4-10-fold (FIG. 3A). Interestingly, when MAZ and E1A were cotransfected together the effect of the two proteins was multiplicative, yielding a 200-fold increase relative to the value observed with vector alone. Similarly, the combination of E1A and Sp1 produced very large increases that approached 200-fold in some experiments.

To confirm that MAZ and Sp1 were being produced from the transfected plasmids, cell extracts for the presence of the epitope-tagged proteins by Western blot assay were analyzed. Both proteins were expressed (FIG. 3B, lane 2, 5), and it was also noted that there was a reproducible strong enhancement of Sp1 expression in cells that also received E1A (FIG. 3B, lane 6). This increase in the level of Sp1 may contribute to the reporter activation detected in cells transfected with E1A plus Sp1. Expression of E1A had negligible effects on the level of MAZ protein expression (FIG. 3B, lane 4).

The steady state level of luciferase RNA (FIG. 3C) was measured to be certain that the activation by MAZ or Sp1 and the combined effect with E1A was due to increased RNA accumulation from the MLP. Quantification of total RNA from transfected cells by hybridization and nuclease S1 digestion produced results that were in good agreement with the results from the transient expression assays. Luciferase RNA levels were undetectable in cells transfected with the reporter gene and the empty expression vector (FIG. 3C, lane 2). Similarly, cotransfection with E1A alone or Sp1 alone did not provide the necessary level of stimulation to detect luciferase RNA (FIG. 3C, lane 4, 6). This was consistent with the transient assays that indicated that E1A or Sp1 alone activated the reporter to a relatively modest extent (FIG. 3A). The stimulation by MAZ was greater in the luciferase assay and this was also true for detection of the mRNA (FIG. 3C, lane 3). A band of about 75 nucleotides is clearly evident and the size is consistent with correctly intiated mRNA derived from the MLP-luciferase expression plasmid. Furthermore, just as predicted from the luciferase assays, the combined effects of MAZ plus E1A or Sp1 plus E1A produced the largest increase in RNA levels (FIG. 3C, lane 5, 7), generating about 34 fold more reporter RNA than when only MAZ was expressed with the reporter gene.

The combined effect of E1A and MAZ or E1A and Sp1 suggested that E1A might interact with these zinc-finger proteins, and an earlier study has shown that Sp1 and E1A can form a complex in vitro (16). To confirm the earlier result with Sp1, and test for the possible interaction of E1A with MAZ, immuoprecipation experiments were performed. Vectors expressing flu epitope-tagged MAZ or Sp1 expression vectors were transfected into HeLa cells in the absence or presence of E1A. Protein from extracts of transfected cells was immunoprecipitated with anti-flu epitope-tag antibody, subjected to electrophoresis in an SDS polyacrylamide gel and blotted to nitrocellulose. Duplicate Western blots were then probed with either anti-E1A or anti-epitope-tag antibody. The antibody to the epitope tag demonstrated that MAZ and Sp1 were immunoprecipitated from the transfected cells (FIG. 3D, left panel). In agreement with earlier in vitro results, the antibody to E1A showed that E1A was coprecipitated with Sp1 (FIG. 3D, right panel, lane 1). In extracts of cells transfected with MAZ and E1A, it is evident that some E1A coprecipitates with MAZ (FIG. 3D, right panel, lane 3), although substantially less E1A is coprecipitated with MAZ than with Sp1. This might indicate that the MAZ-E1A interaction is less stable to the immunoprecipitation conditions than the interaction between E1A and Sp1. However, it is likely that the reduced level of E1A coprecipitated with antibody to epitope-tagged MAZ reflects at least in part the substantially lower level of MAZ expression compared to Sp1 in the transfected cells that received plasmids expressing the transcription factor plus E1A.

### MAZ Activates Transcription Through GC Sequences Flanking the TATA Motif

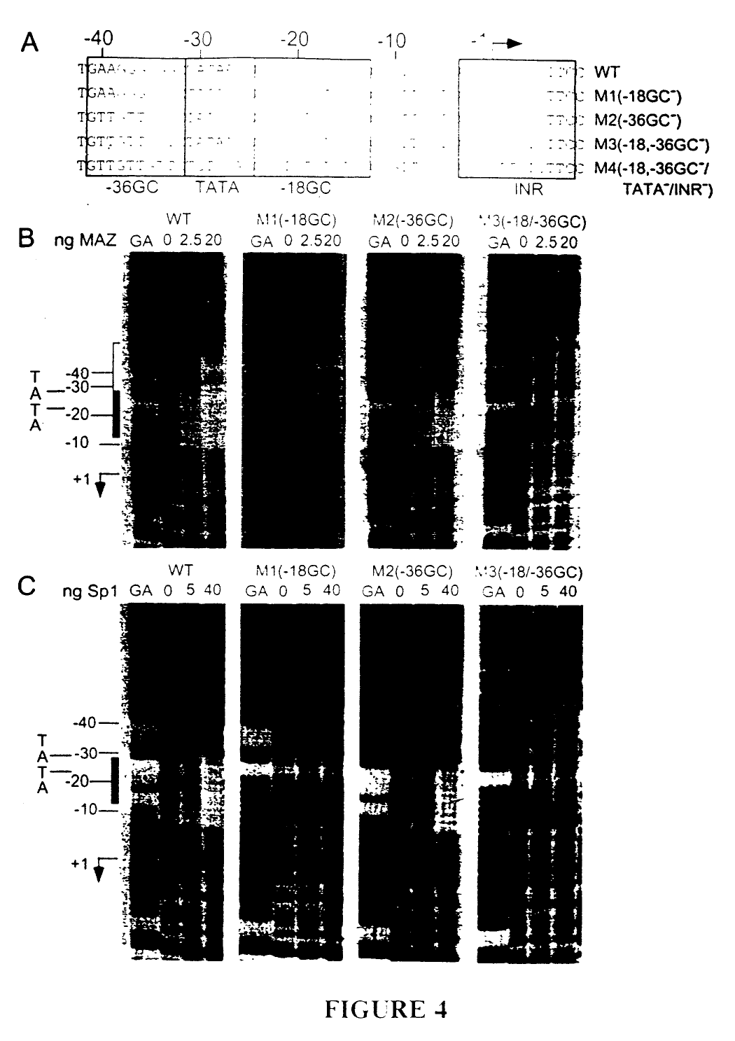

The most intriguing DNA-protein interaction between the MLP and the GC-rich binding factors occurs at the −18GC and −36 GC sequences immediately flanking the TATA box (FIG. 1). The footprints generated by MAZ or Sp1 in this region of the promoter actually span the TATA sequence (FIG. 2). Mutational analysis was performed to ask if these GC-rich sequences participate in the activation of the MLP. A minimal MLP (−45 to +11) that included only the −36 GC sequence, the TATA element, the −18 GC sequence and the initiator region was constructed, and mutant derivatives were produced (FIG. 4A) with multiple base-pair substitutions disrupting the −18 GC motif (Ml), the −36 GC motif (M2), both GC motifs (M3) or both GC motifs as well as the TATA and initiator elements (M4). The effect of the mutations on DNA-protein interactions was examined by footprint analysis (FIG. 4B and C). On the wild type minimal promoter, the pattern of interaction at the −18GC and −36 GC sequences was identical to that observed for the full length promoter; two MAZ binding sites and one Sp1 site were evident. Mutation of the −18GC sequence (M1) reduced the size of the MAZ footprint consistent with disruption of one MAZ binding site, and the M1 mutation completely blocked interaction by Sp1. Thus the −18GC mutation confirms that MAZ interacts with two separate sites in the minimal promoter region and that a single Sp1 binding site is present. The −36GC mutation (M2) reduced the size of the region protected by MAZ, confirming that the −36GC sequence is also a MAZ binding site, but did not alter the Sp1 footprint. The double GC sequence mutation (M3) substantially blocked the ability of both MAZ and Sp1 to interact with the promoter.

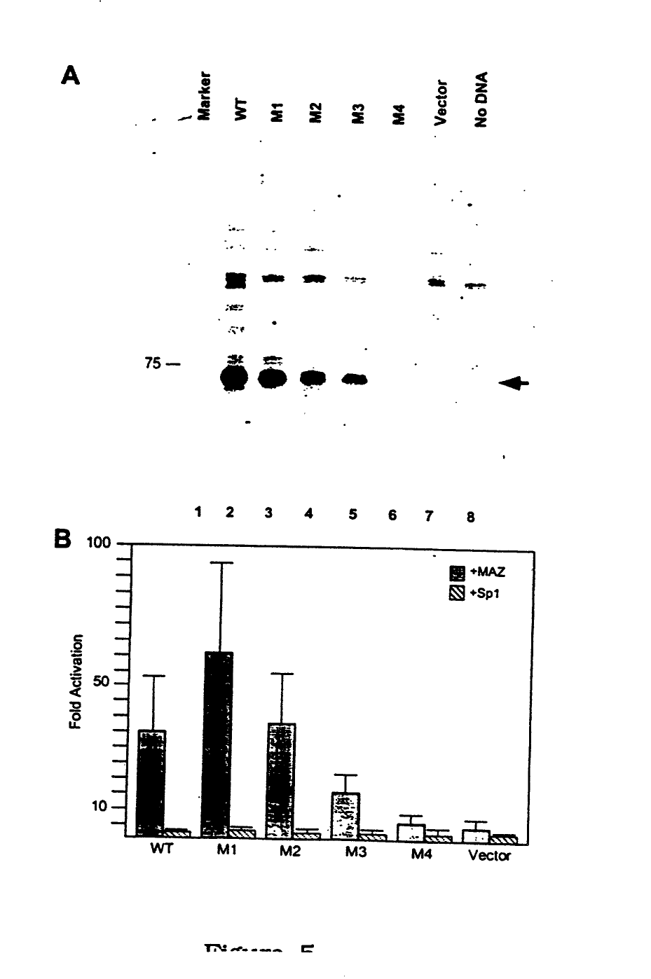

To test the effect of these mutations on promoter activity, supercoiled template DNAs carrying the promoter variants were used to direct in vitro transcription in a whole cell extract, and reaction products were assayed by primer extension. Mutations in the GC sequences reduced the efficiency of transcription (FIG. 5A, lane 2-5). Mutation of the −18 sequence (M1) reduced transcription by a factor of about two relative to the wild type promoter and mutation of the −36 GC sequence (M2) reduced transcription about three fold. Mutation of both GC sequences (M3) produced a more significant reduction of five fold. Transcription reactions programmed with a promoter carrying mutations in both GC sequences, the TATA box and initiator (M4), with the vector without a promoter sequence or with no template DNA did not produce detectable product (FIG. 5A, lane 6-8). The ability of over expressed MAZ and Sp1 to activate the minimal promoter and its mutant derivatives within transfected cells was examined. 293 cells were employed in this assay since they contain the adenovirus E1A protein and both MAZ and Sp1 very strongly activate the MLP in the presence of the viral transcriptional activator (FIG. 3A). Cells were transfected with each MLP construct together with an effector plasmid expressing either flu-epitope tagged MAZ or Sp1. The GC mutations affected activation by MAZ, but had relatively little effect on the modest activation by Sp1 (FIG. 5B). Either single GC mutation (Ml or M2) had little effect on activation by MAZ but when both GC mutations were present (M3) activation by MAZ was reduced to a factor of about 10-15 as compared to 30-50 fold for the wild type minimal promoter. The MLP with mutations in all of its motifs (M4) and the promoterless luciferase plasmid exhibited a 5 fold activation by MAZ. This activation, as well as the consistant 2-3 fold activation of all constructs by Sp1, is probably due to GC-rich sequences in the luciferease vector residing outside of the MLP.

The failure of Sp1 to activate the minimal promoter through the GC sequences flanking the TATA motif (FIG. 5B) suggests that Sp1 acts through its upstream binding site centered at −166 (FIG. 2) to influence transcription of the MLP. Consistent with this proposal, Sp1 cooperated with E1A to strongly activate a reporter that contained this upstream GC element (FIG. 3A).

### Activation of the MLP Residing in the Viral Genome by MAZ and Sp1

To further test the capability of MAZ and Sp1 to activate the MLP, activation of the MLP from within the viral genome was examined. In this case, additional upstream or downstream sequences not present in plasmid constructs might influence activity of the promoter, other viral gene products might impact on its regulation and viral DNA replication could influence its activity. Transfection of the viral DNA molecule, rather than infection with virus, was used so that the effects of added MAZ and Sp1 could be effectively monitored by co-transfection with expression vectors. 293 cells were transfected with adenovirus DNA under conditions that allowed DNA replication to occur or in the presence of hydroxyurea which blocked DNA replication. RNA was harvested at 48h after transfection and analyzed by hybridization with probes that detect RNA encoded by the L1 or L5 regions of the viral genome. L1 and L5 RNAs are both produced from transcripts that initiate at the MLP. In virus-infected cells, L1 RNA is expressed both before and after the onset of viral DNA replication, whereas L5 RNA is produced only after DNA replication begins (reviewed in 17, 22, 27).

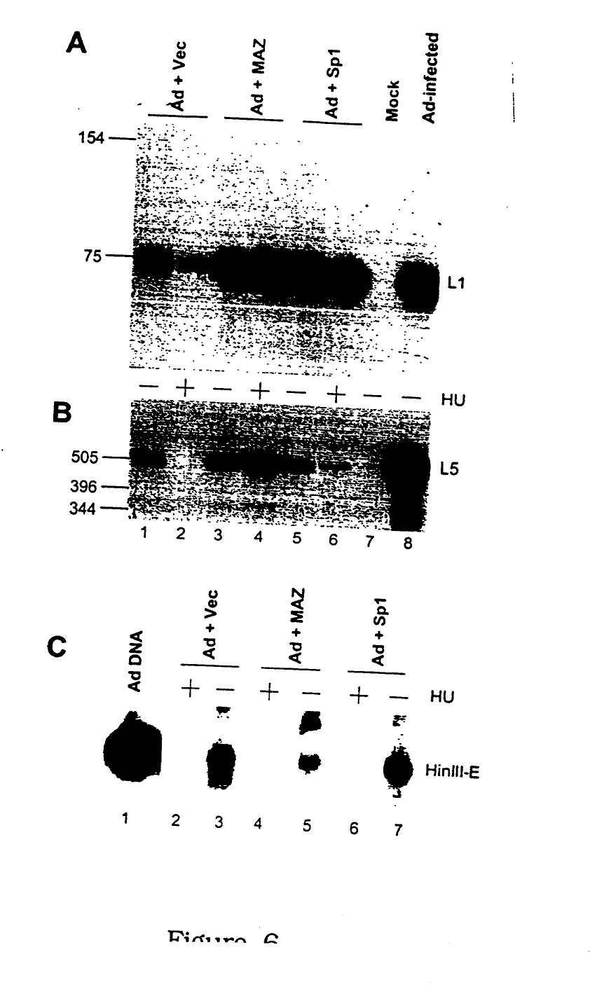

As predicted by the experiments using reporter plasmids (FIG. 3A, 5B), cotransfection of genomic viral DNA with plasmids expressing MAZ or Sp1 stimulated expression from the MLP. The level of L1 RNA was increased 2 to 5 fold by both MAZ and Sp1 (FIG. 6A, lane 1, 3, 5). The addition of hydroxyurea markedly inhibited the accumulation of viral DNA (FIG. 6C) as well as L1 RNA (FIG. 6A, compare lane 1, 2), consistent with the reduced activity of the major late promoter in infected cells before the onset of DNA replication (reviewed in 20). In the presence of the drug, MAZ or Sp1 stimulated the accumulation of L1 RNA by as much as a factor of 17 (FIG. 6A, lane 4, 6). MAZ and Sp1 produced similar effects, and this was consistent with the transient assays using luciferase reporters containing the more complete (−260 to +11) MLP (FIG. 3A). The level of L1 RNA in cells cotransfected with genomic DNA plus MAZ or Sp1 was very high, comparable to the amount that accumulated in 293 cells infected with Ad5 at a multiplicity of 20 pfu/cell (FIG. 6A, lane 8).

The transcription factors also stimulated transcription through the L5 region of the major late transcription unit. L5 RNA accumulation was substantially blocked by hydroxyurea within cells receiving the viral genome without the MAZ or Sp1 expression plasmid (FIG. 6B, lane 2). Hydroxyurea treatment also blocked L5 RNA accumulation in infected cells. This block is consistent with earlier work showing that only the 5′ proximal domain of the major late transcription unit (L1 and L2) is transcribed in the absence of viral DNA replication (reviewed in 17, 22, 27). When MAZ was cotransfected with viral DNA, there was a moderate increase in L5 RNA accumulation in the absence of hydroxyurea and a strong stimulation of L5 RNA accumulation when DNA synthesis was blocked with the drug (FIG. 6B, lane 3, 4). Sp1 did not stimulate L5 RNA accumulation as effectively as MAZ in the absence of DNA replication (FIG. 6B, lane 6), and L5 RNA levels from transfected DNA, even the the presence of MAZ, were substantially less than the levels achieved after infection (FIG. 6B, lane 8). Finally, as a control, activation of the MLP by an expression plasmid that encoded YY1 was tested, another zinc-finger protein (23). There is no known binding site for YY1 in the MLP (10). and, as expected, over expression of YY1 did not influence its expression.

### Discussion

As demonstrated herein, MAZ and Sp1 can bind to the MLP at multiple sites, including GC-rich elements flanking the TATA motif (FIG. 1C). MAZ binds both upstream and downstream of the TATA sequence, whereas Sp1 binds to the downstream but not the upstream site (FIG. 1A). Over expressed MAZ or Sp1 can activate the MLP in transfection assays employing a luciferase reporter with a fairly large segment of the MLP (−260 to +11) (FIG. 2A and C) or in assays where the entire Ad5 genome is transfected into cells (FIG. 6). In contrast, a reporter carrying a minimal MLP (45 to +11) responds to over expressed MAZ, but not Sp1 (Fig. 5B). This suggests that the reporters with a larger segment of the MLP respond to Sp1 through its upstream binding site centered at −166. Genomic footprinting has previously shown that this upstream site is occupied within infected cells (1). Finally, both MAZ and Sp1 cooperate with E1A to induce transcription of the MLP (FIG. 3A and C). Consistent with this cooperation, E1A from extracts of transfected cells can be co-immunoprecipitated with a monoclonal antibody to the epitope-tagged MAZ and Sp1 proteins (FIG. 3D). Earlier work had demonstrated that Sp1 and E1A interact in vitro (16).

Activation of the MLP residing in the viral genome by MAZ or Sp1 was most pronounced when DNA replication was blocked by hydroxyurea (FIG. 6). This may mean that over expression of MAZ or Sp1 can substitute for the MLP activation function normally mediated by DNA replication. So far, the role of DNA replication in the activation of this promoter is unclear (reviewed in 22). Conceivably, MAZ and Sp1 function as a normal part of the transcriptional activation mechanism that depends on DNA replication. Replication might generate genomic templates that are more accessible to MAZ and Sp1 and the increased recruitment of these factors in turn could help to attract the other components of a transcription initiation complex. A higher concentration of MAZ or Sp1, coupled with the delivery of naked DNA to the cell by transfection, might eliminate the need for a more easily accessible template and compensate for the inhibition of DNA replication by hydroxyurea. It was surprising that over expression of MAZ, and to a more limited extent Sp1, enhanced the accumulation of L5 RNA synthesis in the absence of DNA replication (FIG. 6). Normally, DNA replication is a prerequisite for transcription of the distal portion of the major late transcription unit that includes the L5 region, but the mechanism controlling the extent to which the unit is transcribed remains obscure (reviewed in 22). The observation that activation of the MLP in the absence of DNA replication leads to the accumulation of L5 RNA suggests that full length transcription might simply be a mass action effect, i.e., as the promoter becomes more active and more molecules of RNA polymerase begin to transcribe the unit, then more molecules succeed in traveling to the end of the unit, producing L5 RNA.

Yu and Manley (29) examined the transcriptional activity in HeLa whole cell extracts of an extensive set of MLP derivatives containing base-pair substitutions in the GC-rich elements flanking the TATA motif. Several of their variants with multiple G to A transitions in the GC-rich sequences exhibited wild-type activity in the cell-free assay. In contrast, the substitution mutants herein, which prevented MAZ and Sp1 binding to the GC-rich elements (FIG. 4) were somewhat less active (as much as 2.5-fold) than the wild-type minimal MLP. There are several possible explanations for these apparently conflicting results. Different mutations were assayed in the two studies, and it is not known whether the mutations analyzed in the earlier experiments blocked binding of MAZ and Sp1. The different results might also result from the use of different MLP segments in the in vitro transcription assays: the earlier study used a sequence from −66 to +193 and the experiments employed the sequence from −45 to +1 1. Factors that bind within the larger segment of the MLP, but do not have access to the minimal MLP, could obscure the effect of mutations in the GC-rich sequences that flank the TATA motif.

Brunet et al. (3) studied the effects of mutations within the GC-rich elements flanking the TATA motif on the adenovirus chromosome within infected cells. Although multiple G to A transitions in the GC-rich sequence downstream of the TATA element had no observable effect, substitutions in the upstream GC-rich region reduced the activity of the MLP by a factor of 2 to 6. Thus, these results with a minimal MLP (FIG. 5) as well as results of a mutational analysis of the MLP on the viral genome (3), argue that GC-rich sequences adjacent to the TATA motif contribute to the full activity of the MLP.

Do these GC elements contribute to MLP activity by serving as binding sites for MAZ and Sp1? Over expressed Sp1 does not activate a minimal MLP, but it is possible that Sp1 is not limiting in 293 cells, and for this reason added Sp1 does not influence activity of a minimal MLP reporter. Also, other members of the Sp1 family (7, 13) might play a role in the activation. MAZ clearly activates the minimal MLP (FIG. 5B), so it is likely that MAZ and possible that Sp1 family members influence MLP activity through these sequences.

When MAZ is bound to the GC-rich sequences centered at −18 and −36, its DNase I footprint overlaps the TATA motif (FIG. 2 and 4B). Further, when the complex of TFIID/TFIIA/TFIIB interacts with the promoter during the formation of an initiation complex, TFIIA and TFIIB contact the promoter DNA both upstream and downstream of the TATA sequence (5, 18, 26). In the case of the MLP, these contacts would occur within the GC-rich sequences at which MAZ resides. It is possible that MAZ, TFIIA and TFIIB are able to contact these domains of the MLP simultaneously. The attempts to demonstrate a simultaneous interaction of these factors with the MLP have, so far, produced equivocal results. It is also conceivable that, when MAZ interacts with the GC-rich sequences flanking the TATA motif, TBP might be excluded from binding directly to the promoter DNA. In this case, TFIID could be brought to the promoter through protein-protein interactions. It is noteworthy that two single base-pair changes in the TATA motif reduced but did not fully block the expression of properly initiated transcripts from the MLP within infected cells (21). Perhaps TFIID is brought to the promoter exclusively through its interaction with MAZ and Sp1 in this mutant virus. It was previously postulated that MAZ might bring TFIID to promoter sequences in the absence of identifiable TATA motifs in the serotonin la receptor, where MAZ/Sp1 sites are found in close proximity to a series of transcriptional start sites that do not appear to have corresponding TATA elements (20). The potential for MAZ, and perhaps Sp1 family members, to direct TFIID to the major late promoter in the absence of a direct TBP-DNA interaction, raises the intriguing possibility that two alternative mechanisms of initiation might operate at the MLP. One mode of initiation would involve direct binding of TFIID to the TATA motif, and the other would depend on protein-protein interactions to bring TFIID to a promoter containing bound MAZ or Sp1.

### References

1. Albrecht, G., B. Devaux, and C. Kedinger. 1988. Genomic footprinting detects factors bound to the major late and IVa2 promoters in adenovirus-infected HeLa cells. Mol. Cell. Biol. 8: 1534-1539.

2. Ausubel, F. M., R. Brent, R. E. Kingston, D. D. Moore, J. G. Siedman, J. A. Smith, and K. Struhl. 1987. Current Protocols in Molecular Biology. Greene Publishing Associates and Wiley Interscience, New York.

3. Brunet, L. J., L. E. Babiss, C. S. H. Young, and D. R. Mills. 1987. Mutations in the adenovirus major late promoter: effects on viability and transcription during infection. Mol. Cell. Biol. 7: 1091-1100.

4. Chomczynski, P., and N. Sacchi. 1987. Single-step method of RNA isolation by acid guanidinium thiocyanate-phenol-chloroform extraction. Analytical Biochem. 162: 156-159.

5. Geiger, J. H., S. Hahn, S. Lee, and P. B. Sigler. 1996. Crystal structure of the yeast TFIIA/TBP/DNA complex. Science 272:830-836.

6. Graham, F. L. J. Smiley, W. C. Russell, and R. Nairn. 1977. Characteristics of a human cell line transformed by DNA from human adenovirus 5. J. Gen. Virol. 36:59-72.

7. Hagen, G., S. MŜuller, M. Beato, and G. Suske. 1992. Cloning by recognition site screening of two novel GT box binding proteins: a family of Sp1 related genes. Nucl. Acids Res. 20:5519-5525.

8. Harlow, E., B. R. Franza, and C. Schley. 1985. Monoclonal antibodies specific for adenovirus early region 1A proteins: extensive heterogeniety in early region 1A products. J. Virol. 55: 533-546.

9. Harlow, E., P. Whyte, B. R. Franza, Jr, and C. Schley. 1986. Association of adenovirus early-region la peptides with cellular polypeptides. Mol. Cell Biol. 6: 1579-1589.

10. Hyde-DeRuyscher, R., E. Jennings, and T. Shenk. 1995. Functional YY1 binding sites captured from a random sequence pool with a glutathione-S-transferase-YY1 fusion protein. Nucl. Acids Res. 23: 4457-4465.

11. Jones, N., and T. Shenk 1978. Isolation of deletion and substitution mutants of adenovirus type 5. Cell 13: 181-188.

12. Kadonaga, J. T., K. A. Jones, and R. Tjian. 1986. Promoter-specific activation of RNA polymerase II transcription by Sp1. Trends Biochem. Sci. 11: 20-23.

13. Kingsley, C., and A. Winoto. 1992. Cloning GT box-binding proteins: a novel Sp1 multigene family regulating T-cell receptor gene expression. Mol. Cell. Biol. 12: 4251-4261.

14. Kolodziej, P. A., and R. A. Young. 1991. Epitope tagging and protein surveillance. Methods in Enzymology. 194:508-19, 1991. 194: 508-519.

15. Manley, J. L., A. Fire, M. Samuels, and P. A. Sharp. 1983. In vitro transcription:whole cell extract. Methods Enzymology 101: 568-583.

16. Martin, K. J., J. W. Lillie, and M. R. Green. 1990. Evidence for interaction of different eukaryotic transcriptional activators with distinct cellular targets. Nature 346: 147-152.

17. Nevins, J. R., and M. C. Wilson. 1981. Regulation of adenovirus 2 gene expression at the level of transcriptional termination and RNA processing. Nature 290:113-118.

18. Nikolov, D. B., H. Chen, E. D. Halay, A. A. Usheva, K. Hisatake, D. K. Lee, R. G. Roeder, and S. K. Burley. 1995. Crystal structure of a TFIIB-TBP-TATA element ternary complex. Nature 377: 199-128.

19. Parks, C. L., L. -S. Chang, and T. Shenk. 1991. A polymerase chain reaction mediated by a single primer: cloning of genomic sequences adjacent to a serotonin receptor protein coding region. Nucl. Acids Res 19: 7155-7160.

20. Parks, C. L., and T. Shenk. 1996. The serotonin 1a receptor gene contains a TATA-less promoter that responds to MAZ and Sp1. J. Biol. Chem. 271: 4417-4430.

21. Reach, M., L. X. Xu, and C. S. H. Young. 1991. Transcription from the adenovirus major late promoter uses redundant activating elements. EMBO J. 10: 3439-3446.

22. Shenk, T. 1996. Adenoviridae: the viruses and their replication. Fields Virology, B. N. Fields, D. M. Knipe, and P. M. Howely, eds., Lippencott-Raven Publishers, Philadelphia, 2111-2148.

23. Shi, Y., E. Seto, L. -S. Chang, and T. Shenk. 1991. Transcriptional repression by YY1, a human GL1-kruppel-related protein, and relief of repression by adenovirus E1a protein. Cell 67: 377-388.

24. Snyder, R. O., X. Xiao, R. J. Samulski. 1996. Production of Recombinant Adeno-Associated Viral Vectors. Current Protocols in Human Genetics. N. Dracopoli, J. Haines, B. Krof, D. Moir, C. Morton, C. Seidman, J. Seidman, and D. Smith, eds. John Wiley and Sons Publisher, New York, 1-24.

25. Song, B., S. -L. Hu, G. Darai, K. R. Spindler, and C. S. H. Young. 1996. Conservation of DNA sequences in the predicted major late promoter region of selected mastadenoviruses. Virology 220: 390-401.

26. Tan, S., Y. Hunziker, D. F. Sargent and T. J. Richmond. 1996. Crystal structure of a yeast TFIIA/TBP/DNA complex. Nature 381:127-134.

27. Thomas, P. G., and M. B. Mathews. 1980. DNA replication and the early to late transition in adenovirus infection. Cell 22: 523-533.

28. Volkert, F. C. and C. S. H. Young. 1983. The genetic analysis of recombination using adenovirus overlapping terminal DNA fragments. Virology 125:175-193.

29. Yu, Y. -T., and J. L. Manley. 1984. Generation and functional analysis for base-substitution mutants of the adenovirus 2 major late promoter. Nucl. Acids Res. 12:9309-9321.

30. Yu, Y. -T., and J. L. Manley. 1986. Structure and function of the S1 nuclease-sensitive site in the adenovirus major late promoter. Cell 45: 743-751.

31. Shenk, T. 1996. Adenoviruses and their replication. In Virology, eds. Fields, B., Howley, P., and Knipe, D. Raven Press, New York, N.Y., pp. 2111-2148.

What is claimed is:

1. An adenovirus vector comprising the terminal segments of a linear adenovirus genome and a nucleic acid inserted between the terminal segments of the linear adenovirus genome, wherein the terminal segments comprise nucleic acids of the origin of replication and the packaging sequence genes of the adenovirus genome.

2. The adenovirus vector of claim 1, wherein the adenovirus vector is an adenovirus type 5.

3. The vector of claim 1, wherein the nucleic acid is cDNA.

4. The vector of claim 1, wherein the nucleic acid is genomic DNA.

5. The vector of claim 1, wherein the nucleic acid is RNA.

6. The vector of claim 1, wherein the nucleic acid encodes a protein, an antisense RNA, or a ribozyme.

7. The vector of claim 6, further comprising a promoter of RNA transcription operatively, or an expression element linked to the nucleic acid.

8. The vector of claim 6, wherein the promoter comprises a bacterial, yeast, insect or mammalian promoter.

9. The vector of claim 1, further comprising a selectable marker.

10. The vector of claim 9, wherein the selectable marker is beta galactosidase or beta lactamase.

11. A helper adenovirus vector comprising an adenovirus genome having a deletion of the nucleic acid of the origin of replication and the packaging sequence genes of the adenovirus genome.

12. The helper adenovirus vector of claim 11, further comprising a deletion of the E1A gene.

13. The helper adenovirus vector of claim 11, further comprising a deletion of the E1B gene.

14. The helper adenovirus vector of claim 11, further comprising an insertion of one or more nucleic acids of transcription factors within a region of the adenovirus genome.

15. The helper adenovirus vector of claim 14, wherein the transcription factor is MAZ.

16. The helper adenovirus vector of claim 14, wherein the nucleic acid of MAZ consists of sequences from −260 to +11 of the MAZ nucleic acid.

17. The helper adenovirus vector of claim 14, wherein the transcription factor is SP1.

18. The vector of claim 14, further comprising a promoter of RNA transcription operatively, or an expression element linked to the nucleic acid.

19. The vector of claim 18, wherein the promoter comprises a bacterial, yeast, insect or mammalian promoter.

20. The vector of claim 11, further comprising a selectable marker.

21. The vector of claim 20, wherein the selectable marker is beta galactosidase or beta lactamase.

22. A host cell which comprises the vector of claims 1 and 11.

23. The host cell of claim 22, wherein the host is a prokaryotic or eukaryotic cell.

24. The host cell of claim 23, wherein the eukaryotic cell is a yeast, insect, plant or mammalian cell.

25. A pharmaceutical composition comprising the vector of claim 1, the vector of claim 11, and a vector comprising one or more nucleic acids of a transcription factor, and a suitable diluent of carrier.

26. A method of activating adenovirus major late promoter comprising transfecting a cell with the vector of claim 1, the vector of claim 11, and a vector comprising one or more nucleic acids of a transcription factor, thereby activating the adenovirus major late promoter.

27. The method of claim 26, wherein the transcription factor is MAZ.

28. The method of claim 26, wherein the transcription factor is SP1.

29. The method of claim 26, further comprising transfecting the cell with a vector comprising nucleic acid which encodes an E1A gene.

30. A method of preparing virus particles containing a nucleic acid encoding protein of interest comprising transfecting a cell with the vector of claim 1, the vector of claim 11, and a vector comprising one or more nucleic acids of a transcription factor, thereby preparing the virus particles.

31. The method of claim 30, wherein the transcription factor is MAZ.

32. The method of claim 30, wherein the transcription factor is SP1.

33. The method of claim 30, further comprising transfecting the cell with a vector comprising nucleic acid which encodes an E1A gene.

34. The method of claim 30, wherein the cell is a human cell.

35. A gene therapy method comprising administering to a subject a pharmaceutical composition comprising the vector of claim 1 and a suitable diluent or carrier; a pharmaceutical composition comprising the vector claim 10 and a suitable diluent or carrier; and a pharmaceutical composition comprising a vector having one or more nucleic acids of a transcription factor and a suitable diluent or carrier; or a pharmaceutical composition comprising the vector of claim 1, the vector claim 10 and a vector having one or more nucleic acids of a transcription factor and a suitable diluent or carrier, thereby inserting the gene into the subject.

36. The method of claim 35 wherein the transcription factor is MAZ.

37. The method of claim 35, wherein the transcription factor is SP1.

38. The method of claim 35, further comprising administering a pharmaceutical composition comprising a vector comprising a nucleic acid which encodes an E1A gene.

39. The method of claim 35, further comprising administering to the subject a pharmaceutical composition comprising a vector having nucleic acid which encodes an E1A gene and a suitable diluent or carrier.
